(19) United States
(12) Patent Application Publication
(10) Pub. No.: US 2024/0215924 A1
(43) Pub. Date: Jul. 4, 2024

(54) VIDEO-BASED AUTOMATED RECOGNITION OF EPILEPTIC SEIZURE FROM RODENTS IN HOME CAGES

(71) Applicants: City University of Hong Kong, Kowloon (HK); Center for Regenerative Medicine and Hong Kong Health, Hong Kong Institutes of Science & Innovation, Pak Shek Kok (HK)

(72) Inventors: Jufang He, Kowloon (HK); Junming Ren, Kowloon (HK); Yujia Zhang, Kowloon (HK); Zhoujian Xiao, Kowloon (HK); Yujie Yang, Kowloon (HK); Ling He, Kowloon (HK); Lijia Che, Kowloon (HK); Ezra Yoon, Kowloon (HK); Mengfan Zhang, Kowloon (HK); Micky Tortorella, O'Fallon, MO (US)

(21) Appl. No.: 18/093,034

(22) Filed: Jan. 4, 2023

(51) Int. Cl. A61B 5/00; A01K 29/00

(52) U.S. Cl. CPC A61B 5/7264; A01K 29/005; A61B 5/4094

(57) ABSTRACT

There is provided a video-based automated detection method for epileptic seizure behavior of an animal. The method includes providing an epileptic seizure detection dataset for an animal, constructing a deep learning framework by applying a transfer learning with a spatial-temporal network (STN) to localize and detect seizure behavior of the animal based on the epileptic seizure detection dataset, and detecting epileptic seizure behavior of the animal from raw video frames based on the deep learning framework.

### TECHNICAL FIELD

The present invention relates to methods and systems for detecting epileptic seizure of an animal. In particular, the present invention provides video-based automated detection methods for epileptic seizure behavior of an animal, and processors and systems for implementing the methods.

### BACKGROUND

Although the current anti-epilepsy treatment relieves epileptic seizures, many patients with temporal lobe epilepsy (TLE) still suffer from spontaneous recurrent seizures (SRSs) or chronic epilepsy¹⁻⁶. It is urgent to develop new anti-epilepsy drugs (AEDs). The behaviorally observed changes in epileptic seizures of animals are an important metric to assess the efficacy of therapy in AEDs preclinical development⁷⁻¹⁴. However, the long-term manual observation from monitored videos is time-consuming¹⁵⁻²¹. Therefore, researchers are seeking an efficient method to automatically detect seizure behaviors from video recordings.

Recently, some tools based on a deep neural network (DNN) have been widely applied in behavioral analysis of different lab animals and achieved an excellent performance by classifying spatial features from pose estimation or manual definition²²⁻³⁰ However, the detection of epileptic seizures relies on spatial and temporal information because seizure behaviors include syntaxes that are myoclonic seizure (sudden and repetitive movement of head and neck, tail stiffening), clonic seizure (forelimb clonus and rearing), and tonic-clonic seizure (widely running, jumping, and falling)³¹⁻³³. Moreover, it is difficult to detect seizure behaviors with the current methods.

### SUMMARY OF THE INVENTION

The measurement for behavioral changes of spontaneous recurrent seizures (SRSs) in animals is crucial to the preclinical assessment of anti-epilepsy drugs (AEDs). The manual observation for epileptic seizure of chronic model is commonly used but highly laborious and time-consuming.

Preferred embodiments of the invention provide an automatic approach for visual recognition of SRS. The approach may include constructing a large-scale epileptic seizure detection dataset in home-caged mice and using transfer learning with a deep spatial-temporal network (STN) to recognize epileptic seizure behaviors of home-caged mice. The approach could achieve high average precision with 0.97 in validation result by analysis of different network depths and sample durations. Remarkably, the algorithm only takes about 2 minutes to process 24 hours of video data, which drastically increases the processing speed by 175 times compared with human expert observation. Meanwhile, it could achieve excellent detection performance with a small number of epileptic clips (˜150), which can achieve accuracy comparable to human-level. The adaptability of this framework can be extended by recognizing SRS in various species across diverse backgrounds.

According to an aspect of the present invention, there is provided a video-based automated detection method for epileptic seizure behavior of an animal. The method includes providing an epileptic seizure detection dataset for an animal, constructing a deep learning framework by applying a transfer learning with a spatial-temporal network (STN) to localize and detect seizure behavior of the animal based on the epileptic seizure detection dataset, and detecting epileptic seizure behavior of the animal from raw video frames based on the deep learning framework.

In some embodiments, providing the epileptic seizure detection dataset for the animal may include providing a dataset of epileptic mice in home cage (EMHC).

In some embodiments, providing the epileptic seizure detection dataset may include injecting a chemical substance to the animal to induce epileptic seizure behavior, and recording activities of the animal by a camera for a certain period of time to provide video data.

In some embodiments, providing the epileptic seizure detection dataset may further include annotating the video data as epileptic or non-epileptic.

In some embodiments, the video-based automated detection method may be used for preclinical anti-epilepsy treatment evaluation.

In some embodiments, constructing the deep learning framework may include providing the spatial-temporal network (STN) based on pretrained backbones of aggregated residual neural network (ResNeXt) combined with a temporal convolution network (TCN).

In some embodiments, constructing the deep learning framework may include fine-tuning the dataset with the spatial-temporal network (STN) by utilizing a pretrained model trained from a large open human action dataset.

In some embodiments, constructing the deep learning framework may include data augmentation for optimizing training procedure.

In some embodiments, constructing the deep learning framework may include splitting the dataset into three splits training (80%) and validation (20%) and evaluating the performance of the deep learning framework on the validation dataset over different training iterations.

In some embodiments, constructing the deep learning framework may include training individual networks with various sizes of the training set and analyzing the best models in different training proportions of one split.

In some embodiments, the epileptic seizure behavior may be recognized when it is scored as stage 4 or stage 5 in modified Racine scale.

In some embodiments, detecting epileptic seizure behavior of the animal may include detecting seizure events for the animal over a predetermined preclinical test period.

According to another aspect of the present invention, there is provided a processor configured to implement the aforementioned method.

According to yet another aspect of the invention, there is provided a system for implementing a video-based automated detection method for epileptic seizure behavior of an animal, which includes a processor configured to implement the aforementioned method, and a graphic unit interface (GUI) to annotate video data, extract frames and split dataset in dataset construction.

Other features and aspects of the invention will become apparent by consideration of the following detailed description, drawings and claims.

Before any independent constructions of the invention are explained in detail, it is to be understood that the invention is not limited in its application to the details of construction and the arrangement of components set forth in the following description or illustrated in the following drawings. The invention is capable of other independent constructions and of being practiced or of being carried out in various ways. Also, it is to be understood that the phraseology and terminology used herein is for the purpose of description and should not be regarded as limiting.

### BRIEF DESCRIPTION OF DRAWINGS

These and other features of the invention will become more apparent from the following description, by way of example only, with reference to the accompanying drawings, in which:

FIG. 1 is a schematic diagram showing a video-based automated detection method for epileptic seizure behavior of an animal according to an embodiment of the invention.

FIG. 2(a) shows a schematic illustration of KA-induced model according to an embodiment of the invention.

FIG. 2(b) shows observable TLE seizure behavior patterns of the model in FIG. 2(a) based on modified Racine scale.

FIG. 2(c) shows an example architecture of a deep learning framework according to an embodiment of the invention.

FIG. 3(a) shows example video frames representing an example for process of SRS from onset to end.

FIG. 3(b) shows an example confusion matrix illustrating different agreement rates between five observers.

FIG. 3(c) shows a graph of a distribution of seizure frequency based on the seizure duration for human observation.

FIG. 3(d) shows a Precision-Recall curve of a 80% of all dataset for training on a validation set comprising 20% of all dataset in different iterations.

FIG. 3(e) shows an average precision value between validation set comprising 20% of dataset for those n=3 splits evaluated from 0.5 K to 40 K iterations.

FIG. 3(f) shows an example of Grad-CAM with prediction probability in one video prediction of the 80% training model for different iterations.

FIG. 3(g) shows a Precision-Recall curve of different proportions (1%-80% of all data) for training from one split on a validation set comprising 20% of all data.

FIG. 3(h) shows an average precision value between validation sets for those n=3 splits evaluated from 330 to 26,400 training data size (1%-80% of all data).

FIG. 3(i) shows examples of Grad-CAM with prediction probability in one video prediction of the training model for different proportions.

FIG. 4(a) shows schematic illustrations of analysis of feature distribution by t-SNE.

FIG. 4(b) shows schematic illustrations of analysis of feature distribution by t-SNE.

FIG. 5(a) shows comparisons between seizure frequencies of different weeks in seven mice.

FIG. 5(b) shows an average precision value on the decision threshold of 0.5 between generalization videos for those n=3 splits evaluated from 330 to 26,400 training data size.

FIG. 5(c) shows the change of precision and recall value in different threshold strategies.

FIG. 5(d) shows a visual description of decision threshold adjustment.

FIG. 6(a) shows an example of a large-scale unlabelled dataset consisting of different continuous three weeks videos from seven epileptic mice in home cages.

FIG. 6(b) shows the efficiency of the deep learning framework according to an embodiment in the local machine (30×parallelism) and the powerful server (60×parallelism) and human observer.

FIG. 6(c) shows comparisons for the detected epileptic seizure number of different mice between threshold 0.2 (n=508), 0.5 (n=501), 0.8 (n=488) and human observation (n=488).

FIG. 6(d) shows comparisons for missed epilepsy number in different threshold and human observation for three weeks.

FIG. 6(e) shows comparisons of false positive number with threshold 0.5 for induced and non-induced of seven mice respectively.

FIG. 7(a) shows an example frame of epilepsy seizure in a bad video with strong reflection and incorrect direction.

FIG. 7(b) shows the change of cross-entropy loss over iterations fine-tuned by K400 and EMHC.

FIG. 7(c) shows the change of average precision over iterations fine-tuned by K400 and EMHC.

FIG. 7(d) shows example six selected frames from one epileptic mice in chamber environment representing the pattern of overtly behavioral SRS.

FIG. 7(e) shows the change of cross-entropy loss over iterations fine-tuned by K400 and EMHC.

FIG. 7(f) shows the change of precision over iterations fine-tuned by K400 and EMHC.

FIG. 8 shows seizure ethograms for five videos by five different trained annotators.

FIG. 9 shows example special behaviors of a mouse.

FIG. 10 shows example Grad-CAM of three seizure videos for three different mice in experimental chambers.

Before any embodiments of the invention are explained in detail, it is to be understood that the invention is not limited in its application to the details of embodiment and the arrangement of components set forth in the following description or illustrated in the following drawings. The invention is capable of other embodiments and of being practiced or of being carried out in various ways. Also, it is to be understood that the phraseology and terminology used herein is for the purpose of description and should not be regarded as limiting.

### DETAILED DESCRIPTION OF PREFERRED EMBODIMENTS

Hereinafter, embodiments of the present disclosure will be described in detail with reference to the drawings.

There are some tools based on deep neural network (DNN) for behavioral analysis of different lab animals. However, there is a need to provide an advanced method to improve the accuracy of epileptic seizure detection for preclinical drug screening application. Upon investigation on the current behavioral classification algorithms, it is found that the DNN in combination with the temporal convolutional network (TCN) enables to recognize such particular behaviors and could achieve state-of-the-art performance in large-scale open human action datasets³⁴⁻³⁸. This suggests that spatial-temporal network architectures should also greatly improve the accuracy of epileptic seizure detection for preclinical drug screening application.

In embodiments of the invention, there is provided an automated method for direct recognition of seizure behavior itself from arbitrarily long videos in complex environments. To achieve this, in an embodiment, a learning framework is designed (the learning framework can be called “DeepSeizure” hereinafter): DeepSeizure which uses a spatial-temporal network (STN) combined an ResNeXt network with a temporal convolution network (TCN) to localize and detect seizure events directly from raw video frames^{24, 36, 39}. In addition to the construction of this STN, it is developed a pipeline to detect, for the first time, seizure event in home-caged mouse over whole preclinical test period (several weeks) from a single camera. To improve the generalizability, transfer learning and data augmentation to optimize the seizure detection task can be used. Furthermore, the network weights trained by epileptic mice in home cage (EMHC) can be shared, to be directly used to accelerate training of network in new animals of different environments.

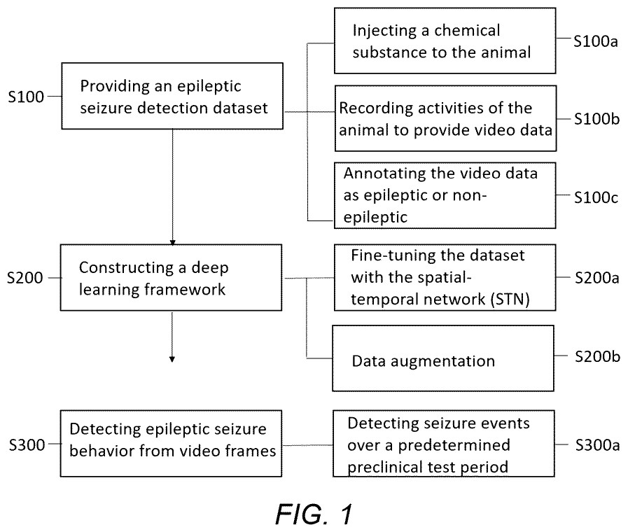

FIG. 1 is a schematic diagram showing a video-based automated detection method for epileptic seizure behavior of an animal according to an embodiment of the present invention. The method includes providing an epileptic seizure detection dataset for an animal (S100), constructing a deep learning framework by applying a transfer learning with a spatial-temporal network (STN) to localize and detect seizure behavior of the animal based on the epileptic seizure detection dataset (S200), and detecting epileptic seizure behavior of the animal from raw video frames based on the deep learning framework (S300).

The step of providing the epileptic seizure detection dataset for the animal (S100) may include injecting a chemical substance to the animal to induce epileptic seizure behavior (S100a), recording activities of the animal by a camera for a certain period of time to provide video data (S100b), and annotating the video data as epileptic or non-epileptic (S100c). The step of constructing the deep learning framework (S200) may include fine-tuning the dataset with the spatial-temporal network (STN) by utilizing a pretrained model trained from a large open human action dataset (S200a), and data augmentation for optimizing training procedure (S200b). The step of detecting epilectic seizure behavior from video frames (S300) may include detecting seizure events for the animal over a predetermined preclinical test period (S300a).

More specific descriptions regarding the video-based automated detection method according to embodiments of the invention will follow with reference to further drawings.

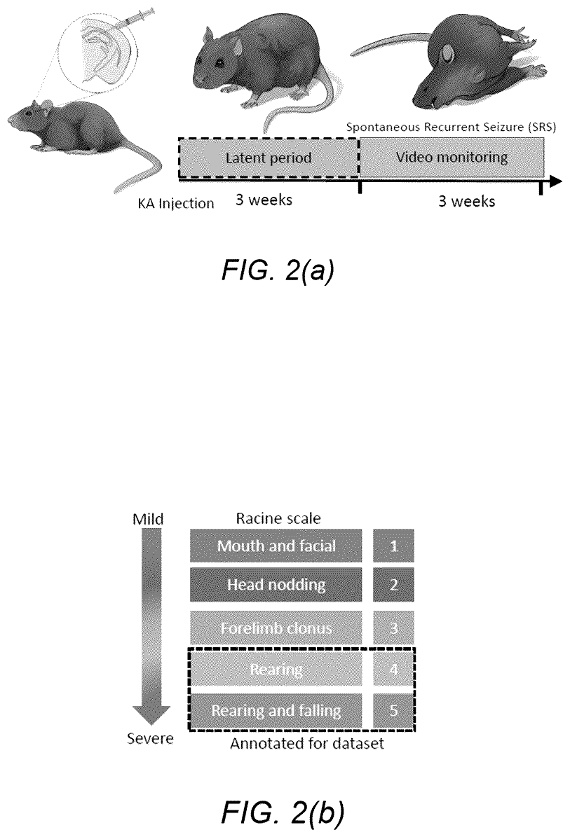

FIGS. 2(a) to 2(c) show schematic illustrations of KA-induced model and a deep learning framework according to an embodiment. In accordance with FIG. 2(a), in order to reproduce key features of chronic TLE in humans, Kainic Acid (KA) is injected unilaterally into dorsal hippocampus of mice. For example, 650 nL KA can be injected into hippocampus CA1 of C57BL/6 mouse to induce TLE seizure behaviors. The SRS emerges after a period of three weeks. Infrared cameras can be utilized to record mice activities in the following three weeks. FIG. 2(b) shows observable TLE seizures including obvious behavior patterns which are scored by modified Racine scale. Generally, seizures lower than stage 4 on the modified Racine scale are hardly observed from videos compared with seizures of stages 4-5. Therefore, the changes of severity 4 or above on the modified Racine scale are focused on more during treatment and drug screening^{8, 24, 49}. In an embodiment, only seizures of stages 4-5 can be recognized as epileptic. Meanwhile, behaviors without seizure activity such as grooming, walking, or eating can be classified as non-epileptic.

FIG. 2(c) shows an example architecture of the deep learning framework (DeepSeizure) according to an embodiment. Cameras are used to record large-scale home cages from front-back view and periodically process data in cameras. The first row shows the pretrained neural network model on large human action dataset K400. Then there are provided the target neural network models on epileptic mice in home cage (EMHC), and epileptic mice in experimental chamber (EMEC) datasets by fine-tuning all network layers of the pretrained K400 model, and the video clips are classified as epileptic or non-epileptic. Here, it is focused on the STN based on the pretrained backbones of aggregated residual neural network (ResNeXt). The network consists of a variant of ResNeXt, whose weights are trained on Kinetics-400. To fine-tune the network for the epileptic detection task, its weights are trained on labeled videos, which consist of epileptic and non-epileptic video clips. During training, the weights are adjusted over iteration so that the network is capable of discrimination between epileptic and non-epileptic in fixed sample duration.

### Benchmarking DeepSeizure

The task of classification with localization for a small free-behaving animal with single view in home cage is challenging. Therefore, to validate the efficacy of the framework, the dataset of epileptic mice in home cage (EMHC) can be constructed. The several key challenges that hindered behavioral recognition during video recording are the refraction due to cage surface; illuminated environment; the frequent action transformation of mice; and distortions from lens limitation. In this embodiment the focus is whether DeepSeizure could deal with these challenges in the raw data without enhancement.

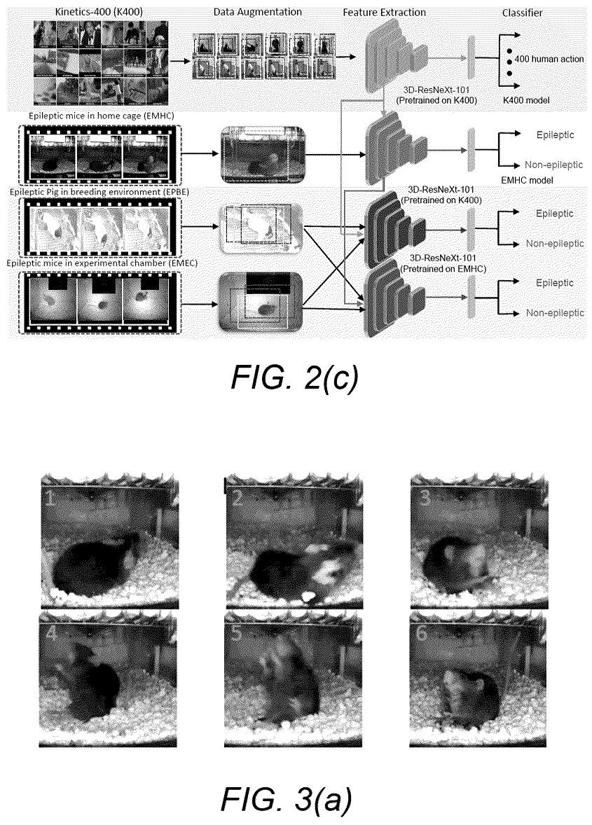

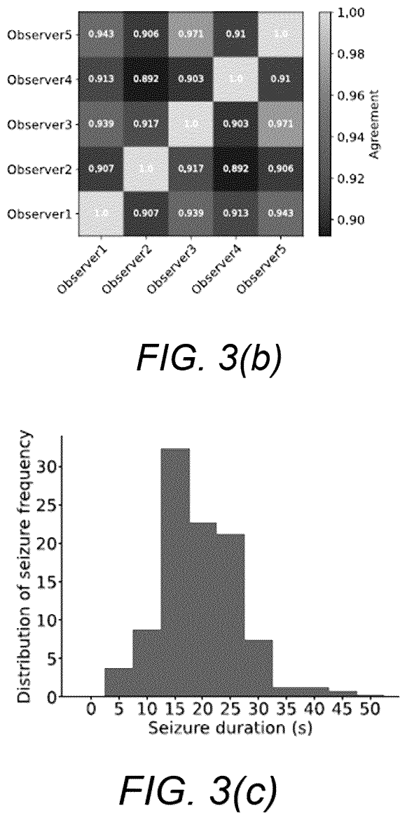

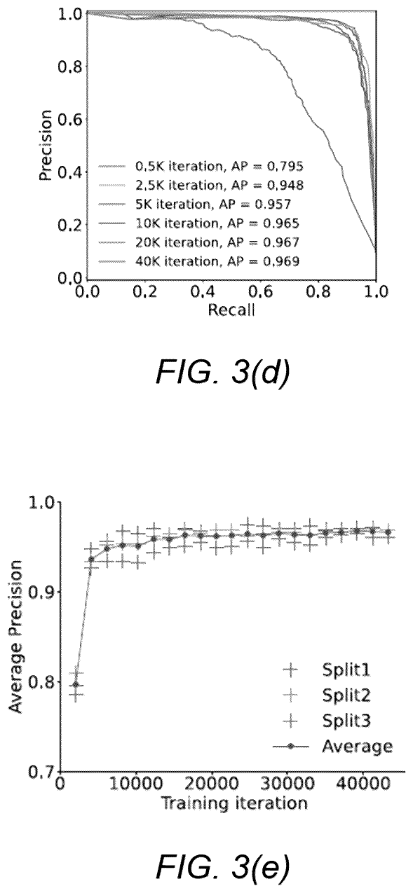

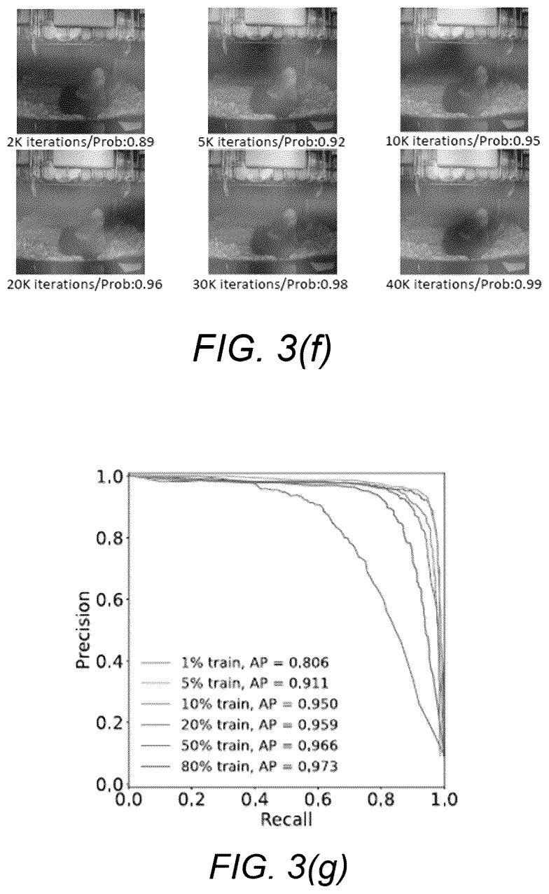

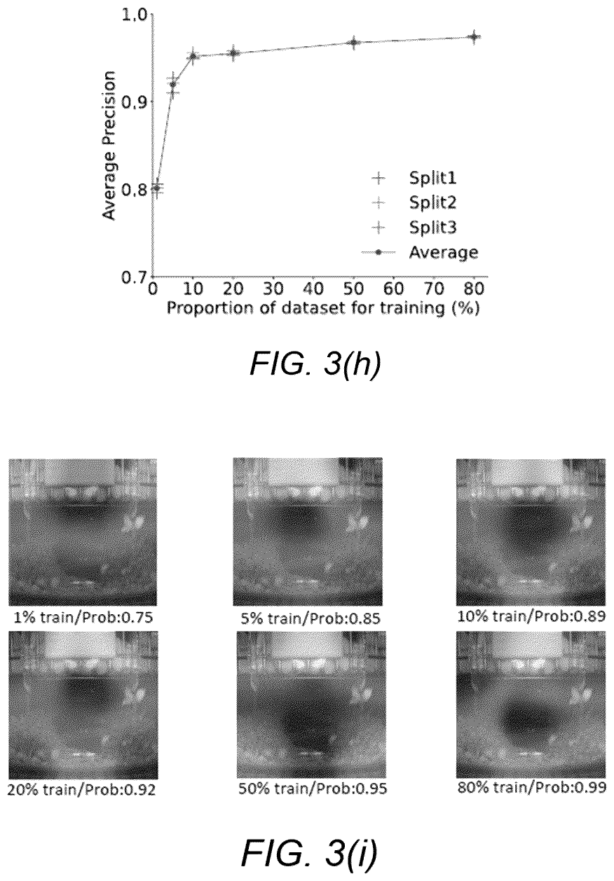

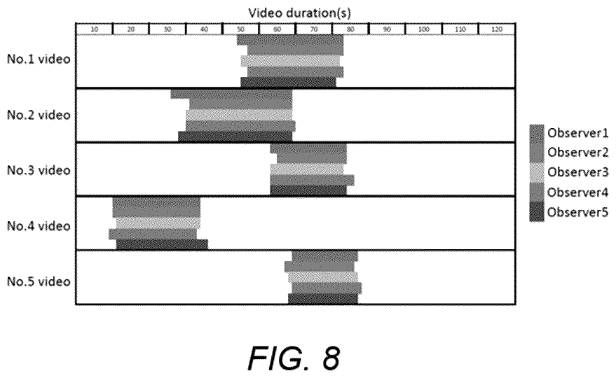

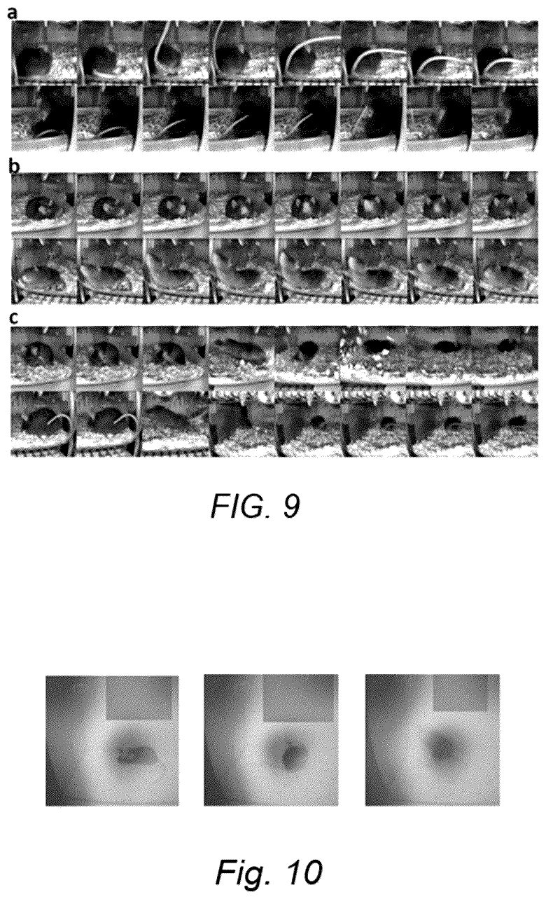

FIGS. 3(a) to 3(i) illustrate evaluation on multiple epileptic mice in home case. FIG. 3(a) shows that six selected frames represent one example for process of SRS from onset to end. Firstly, with reference to FIG. 3(a), five well-trained experts are tasked as annotators to manually label the same five 2 min videos, including epileptic clips according to Racine scale. Each human scorer obtains the same instructions to annotate the onset and end of epileptic behaviors, which will also be described below in section “Methods”. FIG. 3(b) shows a confusion matrix illustrating different agreement rates between five observers in 5 videos and the highest agreement value between observer 3 and observer 5. As shown in FIG. 3(b), a strong average agreement with 92.0% can be found between different human observers when some of them labeled no significant difference in labeled seizure time (see FIG. 8 showing an annotation of SRS behavior and seizure ethograms for five videos by five different trained annotators). Furthermore, to construct the largest epileptic mice dataset, the two annotators with the highest agreement rate (97.1%) can be assigned to extract, for example, 3000 epileptic seizure clips totaling 59,439 seconds (594,393 frames) from 56 epileptic mice models. The distribution of seizure duration is observed to be mainly located in around 15 seconds, as shown in FIG. 3(c). Random trimming can be performed for 30,000 non-epileptic clips with 15 seconds (4,428,917 frames) from the whole video data such as grooming, eating, and drinking.

To quantify the performance of spatial-temporal action detector, the dataset is randomly split into three splits training (80%) and validation (20%), and the performance of DeepSeizure on our validation dataset over different training iterations is evaluated. FIG. 3(d) shows a Precision-Recall curve of a 80% of all dataset for training on a validation set comprising 20% of all dataset in different iteration. To measure the robustness of predictors as compared to human experts, the average precision of the precision-recall curve (see the section “Methods”) is computed and it is found that the recognition model can obtain high average precision close to 97% at the home cage scene, as shown in FIG. 3(d). FIG. 3(e) shows an average precision value between validation set comprising 20% of dataset for those n=3 splits evaluated from 0.5 K to 40 K iterations. The performance for the three different validation set splits can improve over iterations from 79.68±0.7% to 96.9±0.06%, as shown in FIG. 3(e). FIG. 3(f) shows an example of Grad-CAM with prediction probability in one video prediction of the 80% training model for different iterations. Per split result is presented by a cross, and the average is presented by a dot. It is indicated the localization capability of the feature detectors to epileptic mice can become stronger with the increase of training iteration time, by the gradient class activation mapping (Grad-CAM), the method of neural network visualization (FIG. 3(f), see the section “Methods”).

Secondly, 18 individual networks are trained with various sizes of the training set (three splits for 1%, 5%, 10%, 20%, 50% and 80% training set size) and the best models in the different training proportions of one split are analyzed. FIG. 3(g) shows a Precision-Recall curve of different proportions (1%-80% of all data) for training from one split on a validation set comprising 20% of all data. Expectedly, it is observed that the average precision can improve when the number of training samples increases from the precision-recall curve, as shown in FIG. 3(g). For the three set splits, DeepSeizure still achieves an average precision of more than 90%, although the test accuracy attenuates slowly from 80% training set fraction to 5%. Thus, this illustrates that the deep framework could achieve excellent performance using even 150 epileptic clips for training (5%), as shown in FIG. 3(h) which shows the average precision value between validation sets for those n=3 splits evaluated from 330 to 26,400 training data size (1%-80% of all data). Meanwhile, FIG. 3(i) shows examples of Grad-CAM with prediction probability in one video prediction of the training model for different proportions, and the Grad-CAM results may reflect the object localization capability when varies with different proportion of training dataset (see the section “Methods”).

Thus far, these feature detectors work based on the 101-layer of ResNeXt backbone. Other networks with different depth (see Table 1) are also trained and it is found that deeper networks improve the performance with higher average precision on validation datasets (average precision for three identical splits of 80% training set fraction: ResNeXt-18: 75.47±7.12%; ResNeXt-50: 89.52±0.76%; ResNeXt-101: 96.9±0.06%; ResNeXt-152: 70.49±2.51%, average precision mean±s.e.m; see Table 1 below).

TABLE 1
The performance of 3D-ResNeXt in EMHC
dataset of 80% training/20% validation

| | Sample duration | | |
|---|---|---|---|
| Network Depth | 16 frames | 64 frames | 128 frames |
| 18 layers | — | 75.47 | — |
| 50 layers | — | 89.52 | — |
| 101 layers | 90.82 | 96.92 | 93.5 |
| 152 layers | — | 70.49 | — |

It is demonstrated that exceptional performance can be achieved on TLE seizure recognition of EMHC by referring STNs with transfer learning of Kinetics-400 (K400) and small number of epileptic data for training (˜150).

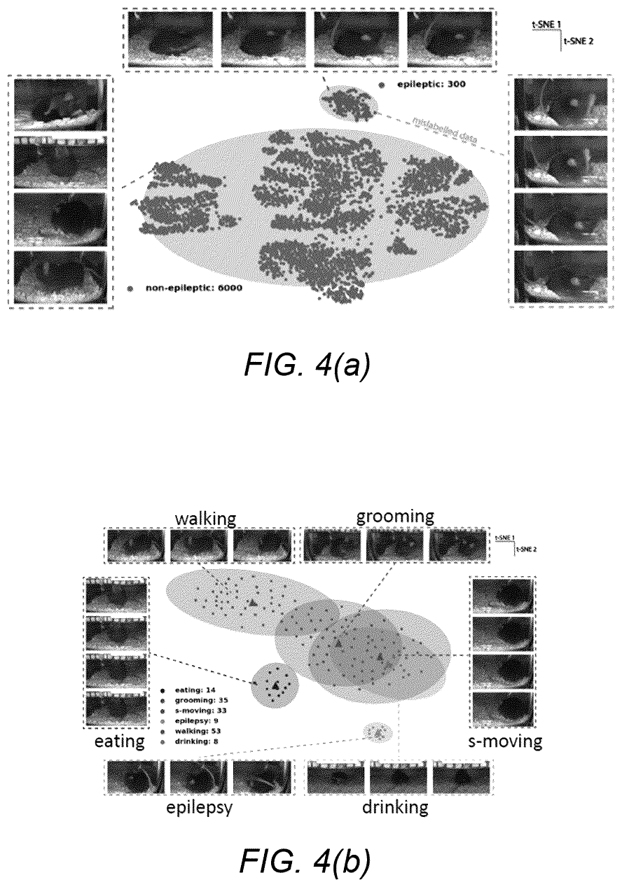

FIGS. 4(a) and 4(b) show schematic illustrations of analysis of feature distribution. To analyze the feature distribution of the data, 6000 non-epileptic and 300 epileptic data are randomly extracted from the EMHC dataset and dimensionality reduction is performed by t-distributed stochastic neighbor embedding (t-SNE) of those videos, as shown in FIG. 4(a). It is observed that the points corresponding to epileptic videos gather and have obvious distance with those representing non-epileptic behaviors in 2D embedding mapping. Meanwhile, it is found that some epileptic samples might be mislabeled as non-epileptic, which facilitates user improvement and fine-tunes the performance of their own dataset. Also, there might be differences between the non-epileptic samples which illustrate the diversity of action features in the dataset. To further investigate the differentiation inside the non-epileptic activities in the dataset, 144 non-epileptic clips can be randomly selected from the dataset and classified with different behaviors which include standing, eating, grooming, s-moving, epileptic seizure, walking, and drinking. Specifically, s-moving represents a sequence of micro-movements for mice to slowly change their positions, as shown in FIG. 4(b) illustrating cluster distribution of different behavioral features by t-SNE. It is observed that different behaviors distribute in different regions in embedding space and each of its centers is far from others. In addition, some behaviors presented overlap with other behaviors which indicates that there is a high similarity of features in those behaviors, like drinking and s-moving. In contrast, it is found that the feature cluster of walking is more distinct than the others. Therefore, the distinguishability of different behaviors in t-SNE space has the potential to classify different behaviors which may help to construct the multi-classification dataset in future.

### Robustness and Threshold Analysis

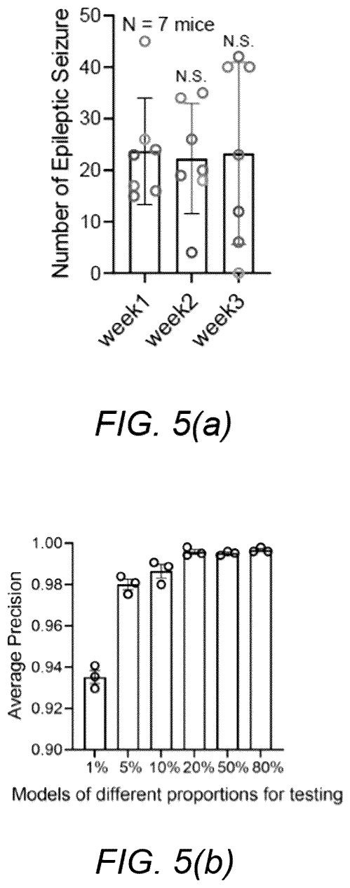

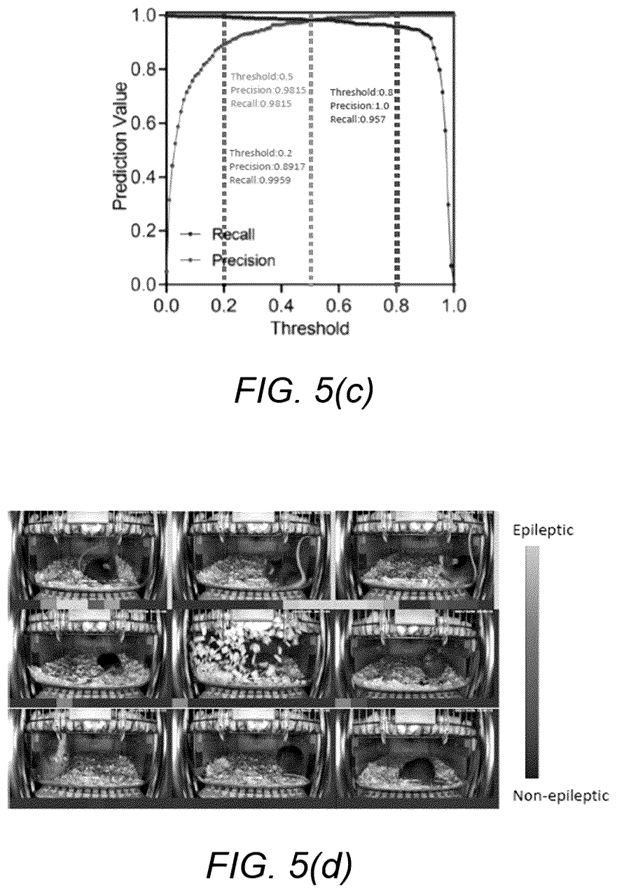

DeepSeizure exhibits the ability to detect epileptic seizures in EMHC dataset. However, whether it can generalize the object never seen before remains ascertained. FIG. 5(a) to FIG. 5(d) illustrate generalization and threshold analysis. FIG. 5(a) shows comparisons between seizure frequencies of different weeks in seven mice (One-way ANOVA, F=0.02, P=0.9791, N.S., not significant). FIG. 5(b) shows an average precision value on the decision threshold of 0.5 between generalization videos for those n=3 splits evaluated from 330 to 26,400 training data size. FIG. 5(c) shows the change of precision and recall value in different threshold strategies. FIG. 5(d) shows a visual description of decision threshold adjustment. We used 10× prediction approach where we predicted one minute video and gave ten viewpoints with 64 frames to each. The proposed model gave the true predicted viewpoints when mice had epileptic seizures or non-seizure activity (first row and third row). While one or several viewpoints can be wrong, the prediction using high threshold of 0.8 improve the performance (second row).

To construct the test dataset, the scorers may find 488 separated epileptic video clips from the data of 7 mice in home cages recorded for three weeks, and randomly select 13646 non-epileptic clips (2,046,900 frames). Furthermore, it is found that the other weeks have a similar frequency of epileptic seizures compared with week 1 (week1: 23.7±3.9, week2: 22.3±4.0, week3: 23.3±6.7, no significant difference, P>0.05), as shown in FIG. 5(a). The best models previously trained by 18 distinct neural networks of the EMHC dataset are utilized to predict the test dataset and it is observed the same increased tendency of average precision from 93.52±0.32% to 99.67±0.07% with training proportions rising, as shown in FIG. 5(b). Besides, no significant changes of performance in the models of EMHC training dataset from 20% to 80% (average precision overall 20%: 99.58%±0.12%; 50%: 99.51%±0.06%; 99.67±0.07%) reveal the strong learning capability of DeepSeizure.

To analyze the effect of different decision thresholds (epileptic prediction probability) on performance, the test result of the best model trained by 80% training set fraction of EMHC is analyzed and recall and precision values with different thresholds: 0.2, 0.5 and 0.8 are obtained, as shown in FIG. 5(c). It is found a high recall (98.15%) and precision (98.15%) in the test dataset when the default threshold value 0.5 is used. The model according to an embodiment can detect more epileptic clips and false positive samples (Green line; Recall:99.59%; Precision:89.17%) in lower threshold of 0.2. In contrast, the higher threshold of 0.8 filters many false positive samples but also filters some epileptic clips (Purple line; Recall:95.69%; Precision: 100%). The performance of threshold adjustment per mouse can be further analyzed. The overall precision increases from 91.4±3.86% to 100% when the threshold increases from 0.2 to 0.8 and the recall of only one mouse is lower than 90% because of small-scale epileptic samples. Some embodiments may select nine videos of one minute including three detected as true positive, ep, three false positives and three true negative clips in the threshold of 0.5 to visualize the description of the decision threshold in the threshold of 0.8, as shown in FIG. 5(d). It is achieved a noticeable decrease in false positive numbers and kept high accuracy of epileptic and non-epileptic detection when the threshold increases to 0.8

Overall, these results demonstrate that proper threshold strategy may achieve an improvement on differentiation between severe seizure and abnormal behaviors, and improve the efficiency of epileptic seizure screening.

### Machine Vs Human Expert in Practical Application

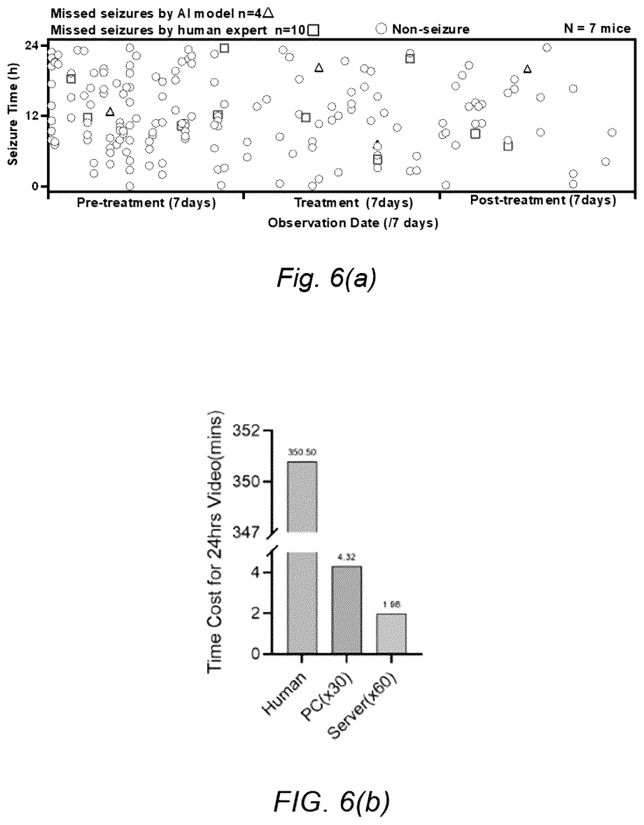

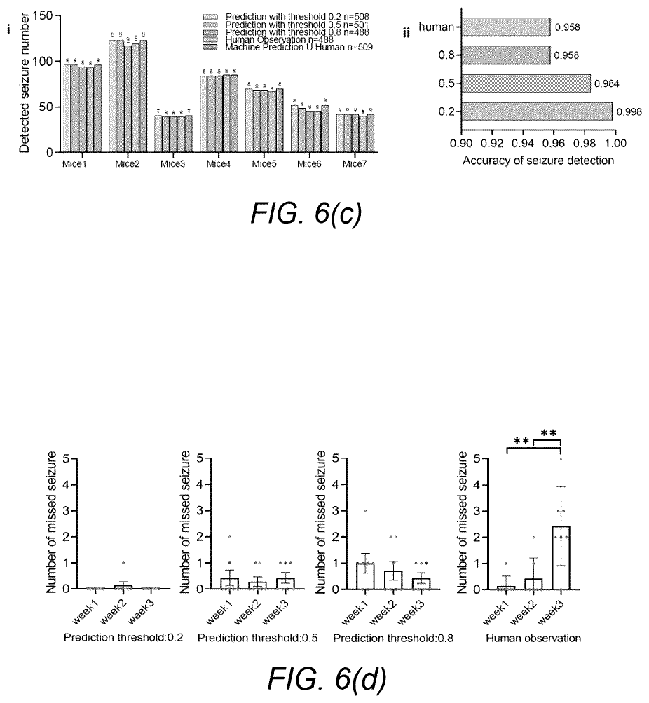

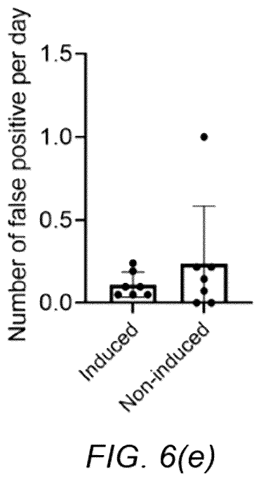

FIG. 6(a) shows an example of a large-scale unlabelled dataset consisting of different continuous three weeks videos from seven epileptic mice in home cages, totaling 147 days (˜3500 hours). The three weeks include seven-day pre-treatment, seven-day treatments by one kind of anti-epilepsy drug, and seven-day post-treatment. Annotators are tasked to annotate epileptic behaviors and count the frequency of seizures from these videos. Meanwhile, the video is tested by the trained models of three splits with 64 frames sampling duration. The preliminary result shows that this anti-epilepsy drug reduced seizure frequency in epileptic mice (FIG. 6(a)). Furthermore, the human expert missed more epileptic seizures (n=10) than the machine (n=4). The hint rate of machines (97.5%) is better than human observation (93.8%). In this proposal, a novel object detection and action recognition network can be combined to improve the robustness of the model in real-scenario applications. Overall, these results indicate that efficiency and accuracy of DeepSeizure outperforms human observers in real-time seizure detection.

FIG. 6(b) shows that the efficiency of DeepSeizure in the local machine (30×parallelism) and the powerful server (60×parallelism) is 87 and 175 times faster than human observer respectively. FIG. 6(c) shows comparisons for the detected epileptic seizure number of different mice between threshold 0.2 (n=508), 0.5 (n=501), 0.8 (n=488) and human observation (n=488). FIG. 6(d) shows comparisons for missed epilepsy number in different threshold and human observation for three weeks (0.2 threshold: one-way ANOVA, F=1, P=0.3874; 0.5 threshold: one-way ANOVA, F=0.7826, P=0.4722; 0.8 threshold: one-way ANOVA, F=0.125, P=0.8833; human: one-way ANOVA, F=10.69, P=0.0009 ***, two-tailed paired t test, week1-week2(P=0.4571, N.S., not significant), week2-week3(P=0.0018, **), week1-week3(P=0.0093, **)). FIG. 6(e) shows comparisons of false positive number with threshold 0.5 for induced and non-induced of seven mice respectively (Two-tailed unpaired t test, P=0.37, N.S., not significant).

To test the efficacy of the framework according to an embodiment in the practical application, 24 hours of continuous video data for three weeks from seven mice, totaling 147 days (˜3500 hours) are detected. The 24 hours of continuous prediction result reveals that the prediction probability of all the seizures reaches higher than 80%, as shown in FIG. 6(a). Meanwhile, some non-epileptic clips incorrectly predicted as epileptic can be filtered by adapting different decisions thresholds. According to an embodiment of the present invention, the algorithm only takes 1.98 minutes to process 24 hours of video data, which increases the processing speed by 175 times compared with human expert observation, as shown in FIG. 6(b). It is observed that both human observers and the model according to an embodiment may miss the epileptic seizure but the model according to an embodiment with different thresholds can obtain higher seizure detection accuracy than human annotators (FIG. 6(c), 0.2: 99.8%; 0.5: 98.4%; 0.8: 95.8%; human: 95.8%). To verify the stability of DeepSeizure, the data of seven mice without the epileptic induction recorded for two weeks can be provided and compared the false positive rate with the epileptic mice group, as shown in FIG. 6(c). It is found that there is no significant difference in false positive frequency between the epileptic induced mice group and non-induced mice group using prediction threshold of 0.5. (Epileptic mice group: 0.37±0.17, non-epileptic mice group: 0.44=0.17, p=0.7661).

Statistical data error during different experimental stages could determine whether the experimental results are real and stable. In preclinical anti-epilepsy research, the majority of researchers focuses on the change of epileptic seizure frequency to validate method feasibility. Thus, the number of missed seizures for three weeks respectively with DeepSeizure and human observers can be analyzed, as shown in FIG. 6(d). It is found that the detector with thresholds of 0.2, 0.5 and 0.8 all have no significant difference in missing seizures compared with week 2 (P>0.5). However, the errors of missing seizures may increase over time and have obvious difference from week 2 to week 3 (** P<0.01). It shows a fluctuation in human results due to varying levels of attention distractions or energy exhaustion, but machine results reflect objective results, as shown in FIG. 6(e).

Overall, these results indicate that efficiency and accuracy of DeepSeizure outperforms human observers in real-time seizure detection.

### Generalization of Varied Environments Learning

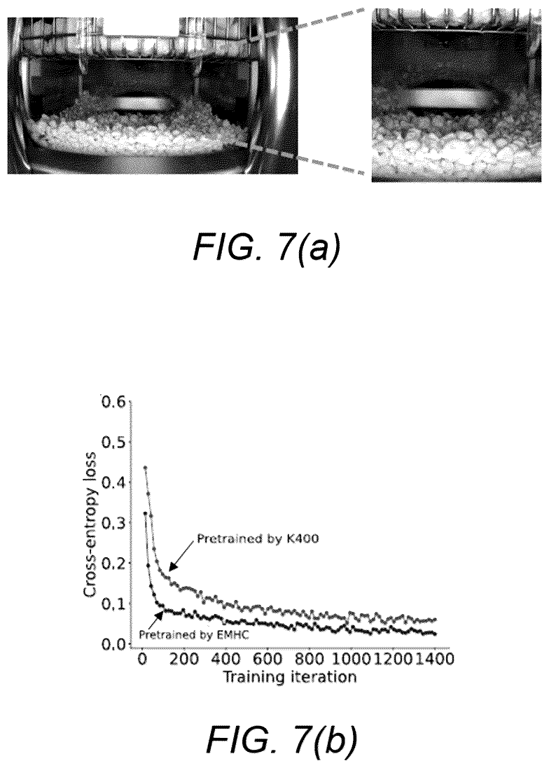

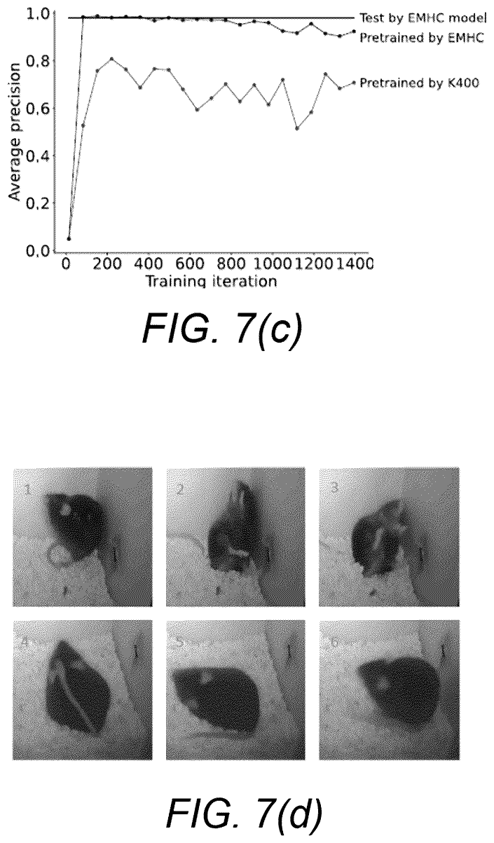

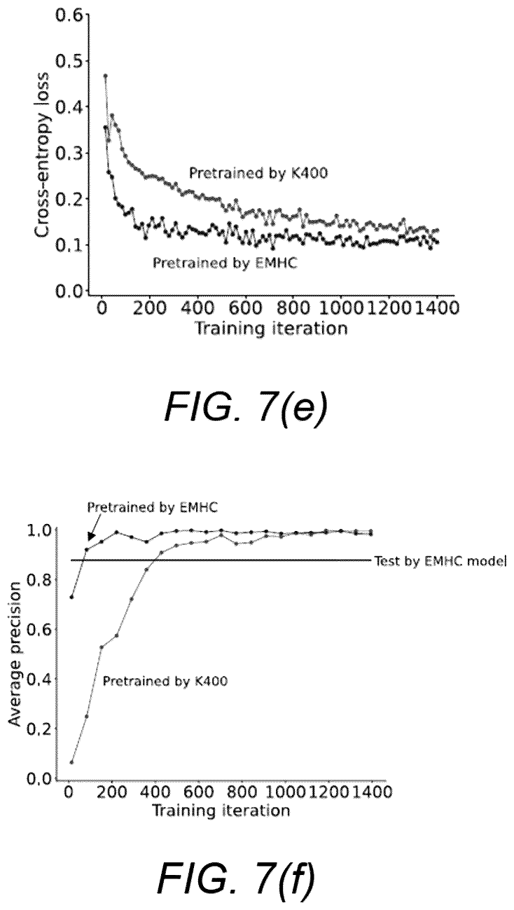

FIGS. 7(a) to 7(f) are provided to explain on evaluation of DeepSeizure in varied background with different transfer learning. FIG. 7(a) shows an example frame of epilepsy seizure in a bad video with strong reflection and incorrect direction. FIG. 7(b) shows the change of cross-entropy loss over iterations fine-tuned by K400 and EMHC. FIG. 7(c) shows the change of average precision over iterations fine-tuned by K400 and EMHC. FIG. 7(d) shows six selected frames from one epileptic mice in chamber environment representing the pattern of overtly behavioral SRS. FIG. 7(e) shows the change of cross-entropy loss over iterations fine-tuned by K400 and EMHC. FIG. 7(f) shows the change of precision over iterations fine-tuned by K400 and EMHC. During the video monitoring, some extreme conditions such as: incorrect positioning of cameras, strong cage reflection and inhomogeneous illumination existed in several epileptic mice models. To test performance of the framework according to an embodiment in extreme data samples, 100 epileptic seizures as well as 1000 non-epileptic clips from one mouse recorded in an extreme environment can be annotated, as shown in FIG. 7(a). Three neural networks are trained based on three splits of 80% data size fraction pretrained on K400 and the performance in test dataset of EMHC is validated. As expected, the overall performance in the test dataset is inadequate (average precision: 80.7±1.2%) with severe training fluctuations, as shown in FIG. 7(c), and was lower than the level of the models trained by 1% of EMHC dataset size fraction (30 epileptic clips) (average precision: 93.52±0.32%, FIG. 7(c)).

The average precision of model increases significantly from 80.7±1.2% to 98.7±0.17%, as shown in FIG. 7(c) when the extreme single mice dataset is directly pretrained by the EMHC model of 80% size fraction. During the training process, the cross-entropy loss pretrained by K400 may decline faster than when pretrained by EMHC model, as shown in FIG. 7(b). It indicates that the features between varied mice behaviors are homogeneous and help severe samples train better.

To further illustrate the generalizability of DeepSeizure, 60 individual epileptic seizure and 600 non-epileptic behavioral clips can be annotated respectively from 6 epileptic mice in experimental chambers, as shown in FIG. 7(d). Then six distinct networks can be trained by three splits for 80% training and 20% validation based on K400 and EMHC, respectively. It is found that the model pretrained by EMHC takes fewer iterations to arrive at better performance levels compared with those trained by K400 from 0 to 800 iterations (K400: 6.38±0.15% to 97.9±0.6%; EMHC: 72.8±10.15% to 98.8±1.16%), as shown in FIG. 7(e). It is also observed that the action predictors precisely localize epileptic mice by Grad-CAM, as shown in FIG. 10. To ascertain the performance of the model trained by EMHC in chamber dataset, the models trained by 80% of EMHC dataset can be used to predict the validation dataset of chamber for three splits. It is found that the performance of EMHC models outperform some models trained by K400 and EMHC (Average precision: 82.5±3.99%; Recall: 75.75±6.06%; Precision: 82.24±6.54%), as shown in FIG. 7(f). This may indicates that the common features of mice in the model could help the dataset of varied backgrounds converge faster.

### DISCUSSION

Chronic epilepsy model mimicked the clinical prototype of human TLE with periodically individual SRS but is not widely used due to time constraint and cost. Current measurement methods of SRS cannot satisfy the demand of large-scale preclinical screening, by localizing the abnormal electroencephalography (EEG) signal.

Firstly, it still needs manual observation for behavioral features to confirm the seizures after EEG signal analysis, demonstrating the non-sufficient automation. And then whether the overtly behavioral seizure after treatment is improved still remains as the gold standard for the drug efficacy even if EEG could detect electrographic-only seizure. Last, it must use much resource for large-scale drug screening because EEG needs heavily equipped environments and multiple channels signal collection. In contrast, the automated behavioral classification according to the embodiments of the present invention could recognize directly the seizures with 3-5 stage in breeding environment by single camera recording. It is efficient and applicable to the large-scale preclinical drug screening in chronic model.

Furthermore, unlike rule-based automated method, DeepSeizure uses the fully data-driven STN to improve on previous single frame methods by extracting temporal information for action detections. Often, it is challenging to train a network in datasets with severely imbalanced class (e.g. epileptic: non-epileptic (1: 10)). The key aspect of the method according to the embodiments of the present invention is to use transfer learning as a training mechanism, which substantially improve the performance with minimum labelling. The seizure detection task can be performed without the constraint of video data such as lighting, resolution, and allows our pipeline to finish end-end detection of raw video without enhancement.

Performance of the model according to the embodiments of the present invention demonstrates the robustness of the deep learning based on STN for epileptic behavioral recognition of mice unseen by our model during training. On the other hand, the model according to the embodiments of the present invention works well on the home cage data from different recording location, which is possible as its strong localizability at an individual level. This is a key advantage for large-scale employment of drug screening in complex environments. The method according to the embodiments of the present invention also exhibits the strong generalizability of long-term epileptic seizure detection in different environments (for example, experimental chamber).

The abnormal behaviors caused by neurological disease especially epilepsy is full complexity and dynamics, by contrast to general behaviors (resting, eating, drinking etc.) that are easily annotated, low-complex and low-dynamics. Thus, time and resources for manual data collection of epileptic behaviors hinders the large-scale data analysis. According to the method according to the embodiments of the present invention, it is scalable, fast and could analyze detailed information from data. The novel combination of data collection method (home-cage recording from front-back view and neuroethology-based expert annotation) and seizure detection method (automated behavioral classifier and transfer learning) can outperform the analysis based on pose-estimation to quantify the postural kinematics.

We do not limit the animals to mice and other animals for example primate in three-dimensional space can be considered. We also do not limit the disease to epilepsy, and other behavioral analysis of depression, Parkinson, and Alzheimer can be considered.

### CONCLUSION

The embodiments provide the utility of the deep learning framework called DeepSeizure in preclinical anti-epilepsy treatment evaluation from different dimensions including performance (comparable human level), scale (dozens to thousands of epileptic videos), experimental subject (mice), and environment (home cage and chamber). Besides, DeepSeizure uses the feature predictors pretrained by different architectures and provides guidelines to (i) automate video trimming based on labels of user-define, (ii) preprocess data such as enhancement or augmentation, (iii) train networks to the desired training set, and (iv) inference for unlabeled videos. Furthermore, the pretrained models based on K400 and EMHC described here could help users to fine-tune or predict their target data in experiments.

The feature detector can simply generalize the unseen epileptic mice from different camera recordings. Furthermore, the performance may change with different decision thresholds of epileptic prediction. In a real-time application, the methods according to the embodiments of the invention can noticeably reduce the duration of processing 24 hours of videos from 350.5 minutes to 1.98 minutes (175 times), outperforming human experts in epileptic detection robustness. The networks pretrained by EMHC can improve generalizability of severely limited training samples of epileptic mice in the experimental chamber (EMEC).

### Method

### Mouse Epilepsy Induced Model

In some embodiments, the KA-induced epilepsy model can be used. Intracerebral KA injection can damage the neurons in the hippocampus. More specifically, KA administration can cause different degrees of hippocampal damage like neuronal loss or neurodegeneration, depending on injection area and dose. This is consistent with the pathological manifestations of TLE seizure patients. Therefore, the KA-induced chronic epilepsy model is adopted. Our data is based on C57BL:6 mice aged 6-10 weeks in experiment. The crucial step is, after anesthetizing the mouse with pentobarbital, 650 nL of KA at a concentration of 0.3 mg/mL is injected in the CA1 region of right hippocampus with an injection speed of no more than 50 nL/min. The injection coordinates are anteroposterior (AP)=−2.06 mm, mediolateral (ML)=−1.80 mm, and dorsoventral (DV)=−1.65 mm, as shown in FIG. 2(a). The animals are returned to their cages after recovery from anesthesia. The intracerebral injected mouse goes through early symptomatic seizures with continuous abnormal behaviors for one to two weeks due to hippocampus neuronal degeneration or inflammation. The spontaneous recurrent seizure (SRS) of these mouse models after the transition period is recorded. All experimental procedures have been approved by the Animal Subjects Ethics Sub-Committee of the City University of Hong Kong.

### Data Collection and Annotation

The mice are fed in a standard ICV cage (size: 45 cm*30 cm*25 cm) of the Laboratory Animal Research Unit (LARU) where an environment with 12/12 h dark and light cycle, constant climatic conditions including temperature, humidity, sterility is provided. In addition, these mice are fed with free access to food and water by professional staff in LARU and kept in healthy condition with free behaviors, during which all procedures are acted in compliance with the regulation of City University Animal Welfare and Ethical Review Body.

In some embodiments, to collect video data of large-scale epileptic mice in home cage, 1080 P high resolution camera can be used to individually record single mice with single view (from front to back) for 24 hours. For experimental chambers recording, the mouse can be placed and recorded with single view (from front to back) by 1080 P high resolution camera in an acrylic test box (size) in a light-and-sound proof chamber for two hours per day.

In some embodiments, five experimented annotators who observed epileptic behaviors for at least half year can be assigned to annotate the time of epileptic behavioral emergence from the same five 2 min videos (10 min total, FIG. 10). From the pool of five annotators, the two annotators with the highest agreement rate are tasked to annotate each video in all datasets. The agreement rate is computed as:

$${R}_{{A}_{1} {A}_{2}} = \frac{{T}_{{A}_{1}} ⋂ {T}_{A 2}}{{T}_{{A}_{1}} ⋃ {T}_{A 2}} \qquad ( 1 )$$

The label of classification is epileptic or non-epileptic. Epileptic seizure of four and five degrees can be defined as epileptic according to the behaviors and severity of Racine scale. For non-epileptic clips, many videos can be randomly extracted from the data excluding epileptic clips and trimmed with similar duration to the epileptic clips. And then, it is checked if these trimmed clips are normal behaviors of mice and include different behaviors such as grooming, walking, and eating.

### Neural Network Implementation

In some embodiments, transfer learning technique is proposed to initialize weight of our neural network instead of training from scratch. The big pretrained model trained from the large open human action dataset which includes over 65M frames and 400 human action labels, kinetics-400, can be utilized to fine-tune our own dataset with the same neural network. The fine-tuned model out-perform both algorithms trained from scratch. Different proportion of the training dataset can be tried to show the better performance with bigger size of data during process of machine learning. Based on the Precision-Recall (PR) curve performance on a per-video basis, it is found that performance is not uniform across all videos.

To generate a training sample, the common practice of data augmentation method in [1, 2] can be applied. For example, a frame in a video can be randomly selected by uniform sampling in time. Then, a 64-frame video clip taking the selected frame can be generated as the start. The video clip is looped as many times as necessary if it is shorter than 64 frames. Next, a location can be randomly chosen from four corners and the center

$$\{ 1 , \frac{1}{{2}^{\frac{1}{4}}} , \frac{1}{\sqrt{2}} , \frac{1}{{2}^{\frac{3}{4}}} , \frac{1}{2} \} .$$

in space with a random spatial scale. The scale is selected from Scale represents the ratio of width of cropped patch to the short side length of the frame. The sample is randomly cropped at positions and scales with aspect ratio 1 and then resized to 112×112. The generated sample is 3×64×112×112, where 3 denotes the RGB channel. Following the previous methods, we subtract the means and divide the variance of ActivityNet in the channels. During training, the samples also can be randomly flipped with 0.5 probability. Identical class labels are maintained for all generated samples as for their original videos

According to the embodiments of the invention, all datasets are trained by STNs based on ResNeXt backbone for 100 epochs and the mini-batch Stochastic gradient descent (SGD) is utilized to optimize the training procedure (learning rate: 1e-1; weight decay: 1e-5; momentum: 9e-1; batch size: 64).

The mice TLE prediction model formulation can follow conventional action recognition methods⁴⁰⁻⁴². We set X={Xᵢ}, i∈[1, N] the mice TLE training dataset, where N is the number of the mice videos in the training set. Let Xᵢ={xᵢ₁, xᵢ₂, . . . , X_{iG}} denotes iₜₕ video consisting of G non-overlapping clips with certain frames. [symbol] (xᵢⱼ; W) represents the function of the mice TLE prediction model with the parameters W on an input Xᵢⱼ. The output of the model is sᵢⱼ={sᵢⱼ¹, sᵢⱼ², . . . , sᵢⱼ^{c}}, where sᵢⱼ^{c} is the prediction score of Cₜₕ class and C is the number of classes. We adopt Softmax function S to normalize the output prediction of F, which can be formulated as

$${\overline{s}}_{ij}^{c} = \frac{{e}^{{s}_{ij}^{c}}}{{\sum }_{k = 1}^{C} {e}^{{s}_{ij}^{k}}} \qquad ( 2 )$$

where s̅ᵢⱼ^{c} is the normalized score of sᵢⱼ^{c}.

Cross entropy (CE) loss can be used for the loss function of the mice TLE prediction model, which is formulated as

$$ℒ ( y , x , W ) = - {\sum }_{k = 1}^{C} {y}_{k} log {k}_{} ( ( x ; W ) ) \qquad ( 3 )$$

where one-hot vector y=(y₁, . . . , y_{C})^{T} is the ground truth label for the input x, [symbol] ₖ represents the function of [symbol] on kₜₕ class.

### Statistical Tests

In the task, the sample sizes between epileptic and non-epileptic clips may have extreme imbalance. It could cause the emergence of so many false positive in daily detection. Therefore, the area under curve (AUC) of PR curve by average precision (AP) is calculated to indicate whether our models could correctly detect all positive samples without incorrectly marking so many non-epileptic as epileptic33. AP is computed as:

$$AP = {\sum }_{n} ( {R}_{n} - {R}_{n - 1} ) {P}_{n} \qquad ( 4 )$$

where Rₙ and Pₙ are the recall and precision respectively at nth threshold. The pair (Rₙ, Pₙ) represents an operating point in the PR curve.

Grouped data are expressed as mean±SEM (standard error of mean). Statistical comparisons namely one-way ANOVA with Bonferroni's multiple comparisons test are performed with GraphPad Prism (version 9.3.0). Statistical significance level can be set at *p<0.05, ** p<0.01, *** p<0.001.

### Gradient-Weighted Class Activation Mapping (Grad-CAM)

To better visualize the model weights on epileptic seizure, Grad-CAM can be used to further interpret the network⁴³. Using Grad-CAM on epileptic data as example, the partial derivative of pixels in the network last layer will be calculated as below

$$\frac{\partial {y}^{epi}}{\partial {A}_{ij}^{k}} \qquad ( 5 )$$

where y^{epi} is the probability output of epileptic data in softmax layer and Aᵢⱼ^{k} is the ith height, jth width pixel in feature map k.

Then, summing up partial derivative of every pixel and performing the global average pooling on it, the sensitive of specific feature map can be obtained. It's given by

$${α}_{k}^{epi} = \frac{1}{Z} {\sum }_{i} {\sum }_{j} \frac{\partial {y}^{epi}}{\partial {A}_{ij}^{k}} \qquad ( 6 )$$

where Z is the number of pixels in feature map k.

Finally, using the calculated sensitive αₖ^{epi} as weight to combine all the feature map linearly and applying the ReLU to retrieve only positive related features, which is computed as

$${L}_{Grad - CAM}^{epi} = ReLU ( {\sum }_{k} {α}_{k}^{epi} {A}^{k} ) \qquad ( 7 )$$

### Interface

DeepSeizure can be developed based on Python and Pytorch (a Python-based deep learning framework) and can be both employed on the platforms of Windows and Linux under the Conda environment. This function is composed of three main parts: dataset construction, training and inference.

A graphic unit interface (GUI) can be provided to annotate users' videos, extract frames and split dataset in dataset construction. Users are allowed to train and validate their own dataset with using the pretrained models by configuration of parameters. Furthermore, DeepSeizure can provide users different models trained by the EMHC dataset to test their video data.

The model according to embodiments of the invention is focusing on video-based automated epilepsy seizure detection in the preclinical development of AEDs. The embodiments of the invention provide the whole workflow of the model, including data annotation, preprocessing, model training, model validation, and visualization. Besides, a software interface is provided that can accomplish offline end-to-end detection of multiple animal subjects from SD card storage videos. The model could replace researchers to observe and analyze the epileptic seizures of the chronic epileptic models in long-term recording videos. It not only reduces labor costs but also decreases subjective errors. Besides, it will help researchers have a quick screening for the potential anti-epileptic drug by the frequency change of behavioral epileptic seizure, which is beneficial for preclinical drug development and provide strong medical proof.

The embodiments of the invention can provide the following advantages when compared to electroencephalography (EEG) based methods:

1. For safety considerations, using recorded video to detect epileptic seizure does not need to implant any devices into the mouse. It won't change the mouse's physical and mental state. Hence, the detected epilepsy of the method according to the embodiments of the invention is much more reliable than the EEG signal detection.

2. For financial considerations, whereas EEG signal detection needs expensive devices to connect to the scalp and record the neuron activity, the method according to the embodiments of the invention only needs a cheap and common infrared camera to collect the mouse's daily data.

3. For the batch size of experiment considerations, EEG is hard to extend the batch size of the experiment since the EEG device is very complex and needs a large space. On the other hand, the method according to the embodiments of the invention only needs one more camera and cage per mouse which is very easy to extend.

4. For experiment duration considerations, the method according to the embodiments of the invention can record the whole daily action of mice and automatically detect the number and duration of daily epileptic seizures. However, the EEG-based method is not practicable to record the whole 24 h signal and keep this 24 h record for a long time (for instance, 3 months).

Nowadays, there has been no seizure recognition method that is focused on experimental animals. Besides, compared to those human epileptic seizure recognition, the method according to embodiments of the invention is validated by a large dataset, which contains 50 mice, a few months 24 h videos, and at least 1000+ epileptic seizure videos. At the same time, those human epileptic seizure recognition is only validated by 100+ epileptic seizure videos, and such method is based on the class-balance dataset, which is different from the real seizures distribution as the data imbalance of seizures and normal daily actions. The performance obtained by the embodiments of the invention is better than lots of the human epileptic seizure recognition methods. The method according to the embodiments of the invention reaches 95+% recall and 50+% precision, and this performance is comparable to human expert.

It should be understood that the above only illustrates and describes examples whereby the present invention may be carried out, and that modifications and/or alterations may be made thereto without departing from the spirit of the invention.

It should also be understood that certain features of the invention, which are, for clarity, described in the context of separate embodiments, may also be provided in combination in a single embodiment. Conversely, various features of the invention which are, for brevity, described in the context of a single embodiment, may also be provided or separately or in any suitable sub-combination.

### REFERENCE

Each of the following references (and associated appendices and/or supplements) is expressly incorporated herein by reference in its entirety:
1. Deckers, C., et al., Current limitations of antiepileptic drug therapy: a conference review. Epilepsy research, 2003. 53(1-2): p. 1-17.
2. Devinsky, O., et al., Changes in depression and anxiety after resective surgery for epilepsy. Neurology, 2005. 65(11): p. 1744-1749.
3. Curia, G., et al., The pilocarpine model of temporal lobe epilepsy. Journal of neuroscience methods, 2008. 172(2): p. 143-157.
4. Lévesque, M. and M. Avoli, The kainic acid model of temporal lobe epilepsy. Neuroscience & Biobehavioral Reviews, 2013. 37(10): p. 2887-2899.
5. Lévesque, M., M. Avoli, and C. Bernard, Animal models of temporal lobe epilepsy following systemic chemoconvulsant administration. Journal of neuroscience methods, 2016. 260: p. 45-52.
6. Löscher, W., et al., Drug resistance in epilepsy: clinical impact, potential mechanisms, and new innovative treatment options. Pharmacological reviews, 2020. 72(3): p. 606-638.
7. Williams-Karnesky, R. L., et al., Epigenetic changes induced by adenosine augmentation therapy prevent epileptogenesis. The Journal of clinical investigation, 2013. 123(8): p. 3552-3563.
8. Krook-Magnuson, E., et al., On-demand optogenetic control of spontaneous seizures in temporal lobe epilepsy. Nature communications, 2013. 4(1): p. 1-8.
9. Löscher, W., L. J. Hirsch, and D. Schmidt, The enigma of the latent period in the development of symptomatic acquired epilepsy traditional view versus new concepts. Epilepsy & Behavior, 2015. 52: p. 78-92.
10. Gelinas, J. N., et al., Interictal epileptiform discharges induce hippocampal cortical coupling in temporal lobe epilepsy. Nature medicine, 2016. 22(6): p. 641-648.
11. Patra, P. H., et al., Cannabidiol reduces seizures and associated behavioral comorbidities in a range of animal seizure and epilepsy models. Epilepsia, 2019. 60(2): p. 303-314.
12. Amengual-Gual, M., I. S. Fernández, and T. Loddenkemper, Patterns of epileptic seizure occurrence. Brain research, 2019. 1703: p. 3-12.
13. Lazarini-Lopes, W., et al., The anticonvulsant effects of cannabidiol in experimental models of epileptic seizures: From behavior and mechanisms to clinical insights. Neuroscience & Biobehavioral Reviews, 2020. 111: p. 166-182.
14. Löscher, W., The holy grail of epilepsy prevention: preclinical approaches to antiepileptogenic treatments. Neuropharmacology, 2020. 167: p. 107605.
15. Tan, G.-H., et al., Neuregulin 1 represses limbic epileptogenesis through ErbB4 in parvalbumin-expressing interneurons. Nature neuroscience, 2012. 15(2): p. 258-266.
16. Jeffrey, M., et al., A reliable method for intracranial electrode implantation and chronic electrical stimulation in the mouse brain. BMC neuroscience, 2013. 14(1): p. 1-8.
17. Balzekas, I., et al., Confounding effect of EEG implantation surgery: Inadequacy of surgical control in a two hit model of temporal lobe epilepsy. Neuroscience letters, 2016. 622: p. 30-36.
18. Willems, L. M., et al., Invasive EEG-electrodes in presurgical evaluation of epilepsies: Systematic analysis of implantation-, video-EEG-monitoring-and explanation-related complications, and review of literature. Epilepsy & Behavior, 2019. 91: p. 30-37.
19. Shuman, T., et al., Breakdown of spatial coding and interneuron synchronization in epileptic mice. Nature neuroscience, 2020. 23(2): p. 229-238.
20. Beesley, S., et al., D-serine mitigates cell loss associated with temporal lobe epilepsy. Nature communications, 2020. 11(1): p. 1-13.
21. Lybrand, Z. R., et al., A critical period of neuronal activity results in aberrant neurogenesis rewiring hippocampal circuitry in a mouse model of epilepsy. Nature communications, 2021. 12(1): p. 1-14.
22 Russakovsky, O., et al., Imagenet large scale visual recognition challenge. International journal of computer vision, 2015. 115(3): p. 211-252.
23. He, K., et al. Deep residual learning for image recognition. in Proceedings of the IEEE conference on computer vision and pattern recognition. 2016.
24 Szegedy, C., et al. Inception-v4, inception-resnet and the impact of residual connections on learning. in Thirty-first AAAI conference on artificial intelligence. 2017.
25. Mathis, A., et al., DeepLabCut: markerless pose estimation of user-defined body parts with deep learning. Nature neuroscience, 2018. 21(9): p. 1281-1289.
26 Nath, T., et al., Using DeepLabCut for 3D) markerless pose estimation across species and behaviors. Nature protocols, 2019. 14(7): p. 2152-2176.
27. Maekawa, T., et al., Deep learning-assisted comparative analysis of animal trajectories with DeepHL. Nature communications, 2020. 11(1): p. 1-15.
28 Kane, G. A., et al., Real-time, low-latency closed-loop feedback using markerless posture tracking. Elife, 2020. 9: p. e61909.
29 Giorgio, J., et al., A robust and interpretable machine learning approach using multimodal biological data to predict future pathological tau accumulation. Nature Communications, 2022. 13(1): p. 1-14.
30. Park, H., et al., Deep learning enables reference-free isotropic super-resolution for volumetric fluorescence microscopy. Nature Communications, 2022. 13(1): p. 1-12.
31. Tsverava, L., et al., Long-term effects of myoinositol on behavioural seizures and biochemical changes evoked by kainic acid induced epileptogenesis. BioMed research international, 2019. 2019.
32. Bain, M., et al., Automated audiovisual behavior recognition in wild primates. Science advances, 2021. 7(46): p. eabi4883.
33. Geuther, B. Q., et al., Action detection using a neural network elucidates the genetics of mouse grooming behavior. Elife, 2021. 10: p. e63207.
34 Feichtenhofer, C., et al. Slow fast networks for video recognition. in Proceedings of the IEEE CVF international conference on computer vision. 2019.
35 Jiang, B., et al. Stm: Spatiotemporal and motion encoding for action recognition. in Proceedings of the IEEE CVF International Conference on Computer Vision. 2019.
36 Lin, J., C. Gan, and S. Han. Tsm: Temporal shift module for efficient video understanding. in Proceedings of the IEEE CVF International Conference on Computer Vision. 2019.
37. Li, Y., et al. Tea: Temporal excitation and aggregation for action recognition. in Proceedings of the IEEE CVF conference on computer vision and pattern recognition. 2020.
38. Feichtenhofer, C. X3d: Expanding architectures for efficient video recognition. in Proceedings of the IEEE/CVF Conference on Computer Vision and Pattern Recognition. 2020.
39 Xie, S., et al. Aggregated residual transformations for deep neural networks. in Proceedings of the IEEE conference on computer vision and pattern recognition. 2017.
40. Hara, K., H. Kataoka, and Y. Satoh. Can spatiotemporal 3d cnns retrace the history of 2d cnns and imagenet? in Proceedings of the IEEE conference on Computer Vision and Pattern Recognition. 2018.
41. Tran, D., et al. A closer look at spatiotemporal convolutions for action recognition. in Proceedings of the IEEE conference on Computer Vision and Pattern Recognition. 2018.
42. Carreira, J. and A. Zisserman. Quo vadis, action recognition? a new model and the kinetics dataset. in proceedings of the IEEE Conference on Computer Vision and Pattern Recognition. 2017.
43. Selvaraju, R. R., et al. Grad-cam: Visual explanations from deep networks via gradient-based localization. in Proceedings of the IEEE international conference on computer vision. 2017.

1. A video-based automated detection method for epileptic seizure behavior of an animal, comprising:
providing an epileptic seizure detection dataset for an animal;
constructing a deep learning framework by applying a transfer learning with a spatial-temporal network (STN) to localize and detect seizure behavior of the animal based on the epileptic seizure detection dataset; and
detecting epileptic seizure behavior of the animal from raw video frames based on the deep learning framework.

2. The video-based automated detection method of claim 1, wherein providing the epileptic seizure detection dataset for the animal comprises providing a dataset of epileptic mice in home cage (EMHC).

3. The video-based automated detection method of claim 1, wherein providing the epileptic seizure detection dataset comprises,
injecting a chemical substance to the animal to induce epileptic seizure behavior; and
recording activities of the animal by a camera for a certain period of time to provide video data.

4. The video-based automated detection method of claim 3, wherein providing the epileptic seizure detection dataset further comprises annotating the video data as epileptic or non-epileptic.

5. The video-based automated detection method of claim 1 is used for preclinical anti-epilepsy treatment evaluation.

6. The video-based automated detection method of claim 1, wherein constructing the deep learning framework comprises providing the spatial-temporal network (STN) based on pretrained backbones of aggregated residual neural network (ResNeXt) combined with a temporal convolution network (TCN).

7. The video-based automated detection method of claim 6, wherein constructing the deep learning framework comprises fine-tuning the dataset with the spatial-temporal network (STN) by utilizing a pretrained model trained from a large open human action dataset.

8. The video-based automated detection method of claim 6, wherein constructing the deep learning framework comprises data augmentation for optimizing training procedure.

9. The video-based automated detection method of claim 1, wherein constructing the deep learning framework comprises splitting the dataset into three splits training (80%) and validation (20%) and evaluating the performance of the deep learning framework on the validation dataset over different training iterations.

10. The video-based automated detection method of claim 9, wherein constructing the deep learning framework comprises training individual networks with various sizes of the training set and analyzing the best models in different training proportions of one split.

11. The video-based automated detection method of claim 1, wherein the epileptic seizure behavior is recognized when it is scored as stage 4 or stage 5 in modified Racine scale.

12. The video-based automated detection method of claim 1, wherein detecting epileptic seizure behavior of the animal comprises detecting seizure events for the animal over a predetermined preclinical test period.

13. A processor configured to implement the method of claim 1.

14. A system for implementing a video-based automated detection method for epileptic seizure behavior of an animal, comprising:
a processor configured to implement the method of claim 1; and
a graphic unit interface (GUI) to annotate video data, extract frames and split dataset in dataset construction.
